(19) United States
(12) Patent Application Publication
(10) Pub. No.: US 2021/0145356 A1
(43) Pub. Date: May 20, 2021

(54) MOBILE ENABLED CHEST WORN ECG MONITOR

(71) Applicant: Medicomp, Inc., Melbourne, FL (US)

(72) Inventors: Anthony Balda, Melbourne, FL (US); George Koos, Melbourne, FL (US)

(73) Assignee: Medicomp, Inc., Melbourne, FL (US)

(21) Appl. No.: 17/098,651

(22) Filed: Nov. 16, 2020

Related U.S. Application Data
(60) Provisional application No. 62/935,391, filed on Nov. 14, 2019.

(51) Int. Cl. A61B 5/00; A61B 5/0205

(52) U.S. Cl. CPC A61B 5/4803; A61B 5/0404; A61B 5/0205

(57) ABSTRACT

A system for physiological signal monitoring including a housing, a physiological signal sensor carried by the housing, a microphone carried by the housing, and a symptom button carried by the housing, wherein the microphone is operable to capture an audio recording upon activation of the symptom button and a physiological signal captured by the physiological signal sensor during the activation of the symptom button is associated with the audio recording.

### RELATED APPLICATIONS

This application claims priority under 35 U.S.C. § 120 of U.S. Patent Application Ser. No. 62/935,391 (Attorney Docket No. 612.00139) filed on Nov. 14, 2019 and titled MOBILE ENABLED CHEST WORN ECG MONITOR. The content of this application is incorporated herein by reference.

### FIELD OF THE INVENTION

The present invention relates to systems and methods for heart monitoring and, more specifically, to ECG monitors adapted to be chest worn and mobile enabled.

### BACKGROUND OF THE INVENTION

Heart disease is a leading cause of death in the United States. Some patients may benefit from long-term ECG monitoring outside of a clinical setting. For example, atrial fibrillation and myocardial ischemia may occur episodically. Some episodes may occur without patient symptoms. Myocardial ischemia, if persistent and serious, can lead to myocardial infarction (heart attack). During a myocardial infarction, electrophysiological changes may be detected by an ECG monitoring device. For accurate diagnosis and effective treatment of many episodic heart conditions, medical professionals need to receive accurate and timely information regarding the frequency and duration of such episodes.

In conventional long-term ECG monitoring, such as with continuous Holter monitors or event monitors, mounting of the monitor typically involves preparation of the patient's skin to receive the monitoring device. Chest hair may be shaved or clipped from men. The skin is abraded to remove dead skin cells and cleaned. A technician trained in electrode placement applies the electrodes to the skin with an adhesive. Each electrode of such conventional monitors is attached to an insulated wire that is routed some distance across the patient's body to an amplifier designed to amplify the ECG signal in preparation for further processing. Such monitoring systems are often worn by a patient for up to a month.

Traditional long-term monitoring systems like those described above present a number of problems. During use, the patient must be careful not to pull on the wires connected to the electrodes, lest the electrodes be pulled off the skin. Removing an electrode with its strong adhesive may be painful to the patient. Furthermore, certain types of electrodes require use of a gel next to the skin to improve conductivity at the point of connection of the metal electrode to the skin. Prolonged exposure to the gel can irritate the skin. Additionally, information captured by the device must be provided to external equipment for analysis or monitoring. These and other factors associated with traditional long-term monitoring solutions may discourage a patient from using the ECG monitor as directed by medical personnel.

Alternative health monitoring system designs exist that attempt to address the many shortcomings of traditional ECG monitors.

No device currently exists that supports long term wear of a reusable heart monitoring device with built in cellular communication equipment, which can be power intensive. Consequently, a need exists for increasingly comfortable, convenient, and energy efficient monitoring devices for both personal and medical use, and that overcome the shortcomings of common implementations in the field.

This background information is provided to reveal information believed by the applicant to be of possible relevance to the present invention. No admission is necessarily intended, nor should be construed, that any of the preceding information constitutes prior art against the present invention. This reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### SUMMARY OF THE INVENTION

With the above in mind, embodiments of the present invention are related to a system for physiological signal monitoring including a housing, a physiological signal sensor carried by the housing, a microphone carried by the housing, and a symptom button carried by the housing. The microphone may be operable to capture an audio recording upon activation of the symptom button and a physiological signal may be captured by the physiological signal sensor during the activation of the symptom button and associated with the audio recording.

### BRIEF DESCRIPTION OF THE DRAWINGS

Some embodiments of the present invention are illustrated as an example and are not limited by the figures of the accompanying drawings, in which like references may indicate similar elements.

FIG. 1 is a top plan view of the cellular enabled chest worn heart monitor according to an embodiment of the invention.

FIG. 2 is a bottom plan view of the cellular enabled chest worn heart monitor of FIG. 1.

FIG. 3 is a top plan view of the cellular enabled chest worn heart monitor according to an embodiment of the invention.

FIG. 4 is a perspective view of the cellular enabled chest worn heart monitor of FIG. 3.

FIG. 5 is a perspective view of the cellular enabled chest worn heart monitor according to FIG. 3.

FIG. 6 is a profile view of a cellular enabled chest worn heart monitor according to an embodiment of the invention.

FIG. 7 is a perspective view of the cellular enabled chest worn heart monitor according to an embodiment of the invention.

FIG. 8 is a perspective view of a removable electrode component of the cellular enabled chest worn heart monitor of FIG. 7.

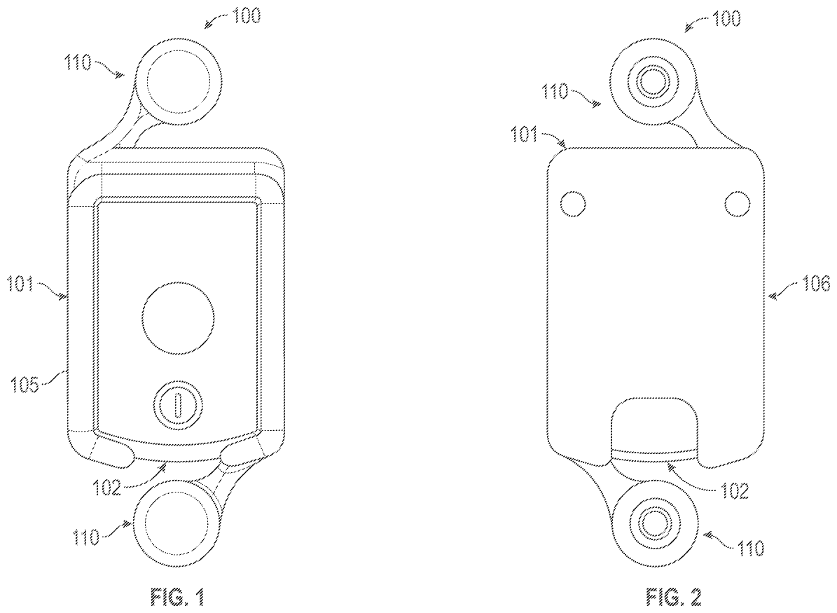

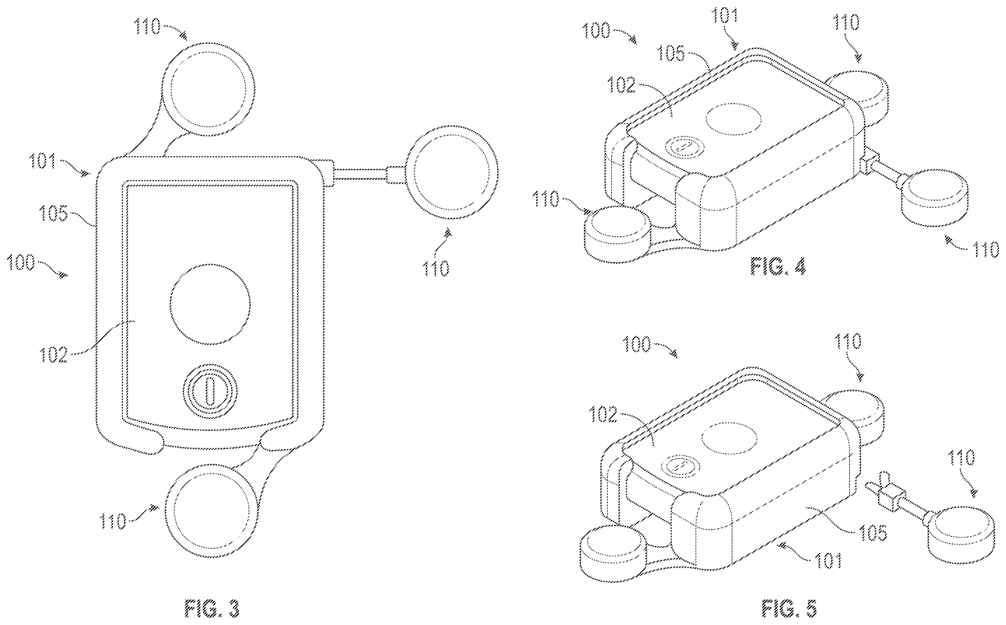

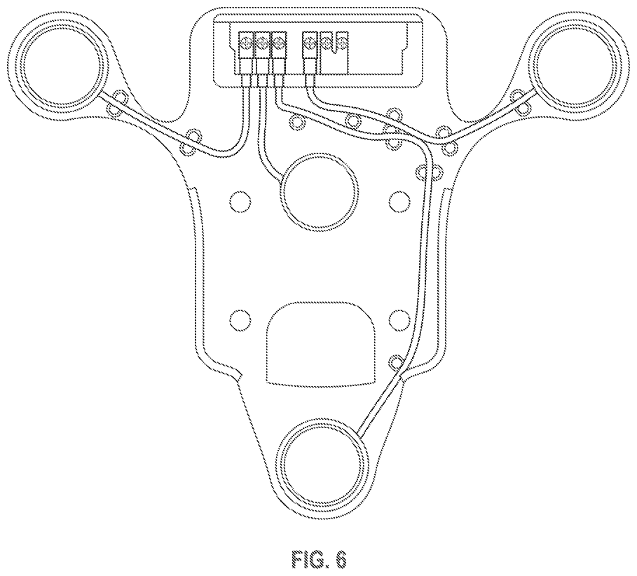

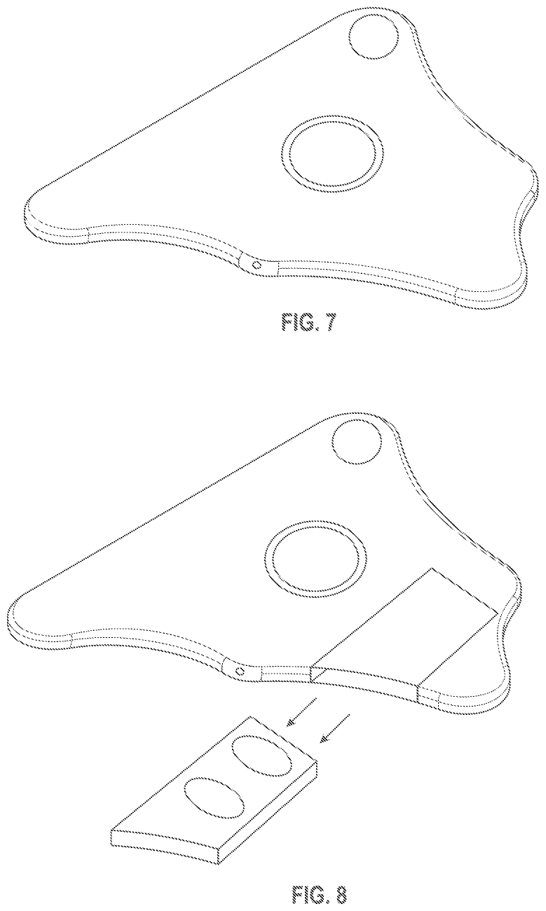

### DETAILED DESCRIPTION OF THE INVENTION

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Those of ordinary skill in the art realize that the following descriptions of the embodiments of the present invention are illustrative and are not intended to be limiting in any way. Other embodiments of the present invention will readily suggest themselves to such skilled persons having the benefit of this disclosure. Like numbers refer to like elements throughout.

Although the following detailed description contains many specifics for the purposes of illustration, anyone of ordinary skill in the art will appreciate that many variations and alterations to the following details are within the scope of the invention. Accordingly, the following embodiments of the invention are set forth without any loss of generality to, and without imposing limitations upon, the invention.

In this detailed description of the present invention, a person skilled in the art should note that directional terms, such as “above,” “below,” “upper,” “lower,” and other like terms are used for the convenience of the reader in reference to the drawings. Also, a person skilled in the art should notice this description may contain other terminology to convey position, orientation, and direction without departing from the principles of the present invention.

Furthermore, in this detailed description, a person skilled in the art should note that quantitative qualifying terms such as “generally,” “substantially,” “mostly,” and other terms are used, in general, to mean that the referred to object, characteristic, or quality constitutes a majority of the subject of the reference. The meaning of any of these terms is dependent upon the context within which it is used, and the meaning may be expressly modified.

An embodiment of the invention, as shown and described by the various figures and accompanying text, provides a cellular enabled chest worn heart monitor 100 including one or more electrodes 101 integrated with a monitoring device 103, and an adhesive surface adapted to secure the monitor 100 to a patient.

The monitoring device 103 of the cellular enabled chest worn heart monitor 100 may be incorporated into a housing that is secured to the patient's body and carries one or more electrodes 101. The housing may have an adhesive layer adapted to secure the housing to a patient's skin. In another embodiment, one or more electrodes 101 may be secured to or integrated with the housing and have an adhesive layer adapted to secure to electrode 101 to the patient allowing the monitoring device 103 to be secured to the patient's body without the need for a separate adhesive patch or an adhesive layer on the housing.

The monitoring device 103 may include a battery or other power source adapted to power the monitoring device 103 while it is worn by the patent. The monitoring device 103 may include photovoltaic cells to provide power to the power source. The monitoring device 103 may include a processor contained within a waterproof or water-resistant housing. In one embodiment, the power source may be removed from the patch 102 and replaced with a charged power source.

The monitoring device 103 may include a microphone adapted to capture the patient's voice documenting symptoms. The audio captured by the microphone may be recorded and stored in a memory of the monitoring device 103. The audio recording may be correlated to ECG data captured contemporaneously with the recording to allow for analysis of the ECG data occurring when the patient experienced the symptoms described. The monitoring device 103 may include a symptom button. Depressing the symptom button may cause the ECG data to be marked to correlate in time to the depression of the symptom button. Depressing the symptom button may activate the microphone and associate the recording with the ECG data measured proximate to the depression of the symptom button.

The monitoring device 103 may include an RFID, which may be utilized to identify the monitoring device 103.

The monitoring device 103 may include removable fingertip electrodes carried in the housing of the monitoring device 103. The fingertip electrodes may be removable from the housing of the monitoring device 103. In such an embodiment, the fingertip electrodes may function to record a physiological signal, including, but not limited to, a heart waveform, when the fingertip electrodes are removed from the housing of the monitoring device 103. The fingertip electrodes may transmit the captured data independently or may provide the data to electronics included on the monitoring device 103 wirelessly or through a wired connection when the fingertip electrodes are carried by the monitoring device 103.

The monitoring device 103 may include a visual indication of an error present in the system. By way of example, and not as a limitation, this visual indication may be provided by an LED or the like. In one embodiment, a visual indication may be presented to the user when no ECG or other physiological signal is detected by the monitoring device 103.

The monitoring device 103 may be capable of communicating on one or more wireless networks, including, but not limited to, a cellular network, Wi-Fi network, or Bluetooth connection.

The cellular enabled chest worn heart monitor 100 may be configured to transmit ECG data on a cellular system. In one embodiment, the monitoring device 103 may transmit the data. One of the challenges inherent in a body worn cellular enabled device 100 is limiting specific absorption rates (SAR) of the device 100, particularly when the monitoring device 103 is worn by the patient and adapted to transmit cellular data while being worn. The difficulty lies in balancing SAR with adequate signal power, antenna design, and mechanical design.

Additionally, the battery life of a cellular enabled device is perceptually directly correlated to the quality of the device. Therefore, it is important to provide robust battery life. Additionally, when the cellular enabled device is an ECG monitor, battery life is also correlated to the patient's ease of use and compliance. Several strategies may be utilized individually or in combination to minimize SAR and increase battery life.

One embodiment of the cellular enable chest worn heart monitor 100, may (1) collect analog ECG signals of a patient's heart activity, (2) convert the analog ECG signals, to digital signals at a chosen sample rate, and (3) transmit the digital signal samples to a monitoring center for further analysis or diagnosis of the ECG signal. The further analysis may result in a diagnosis of the type of arrhythmias that are occurring in the patient. Implementation of such an embodiment may be affected by cellular connectivity and data plans. Redundant communication paths may be established or there may be an attempt to establish redundant communication paths to transmit data from the monitoring device 103 to a monitoring center. Such redundancy may increase reliability of the cellular enabled chest worn heart monitor 100 or allow faster notification to a medical professional of the patient's condition. In the event transmission of the ECG signal to a monitoring center is not available, the ECG signal may be stored locally on the monitoring device 103 and forwarded to a monitoring center at a later time.

One embodiment may implement a selective send protocol. In such a protocol, not all ECG signals, but only certain ECG signals may be transmitted by the cellular enabled device 100. By way of example, and not as a limitation, the monitoring device 103 may identify and detect critical arrhythmia data collected by the electrodes 101. Only the ECG signals containing the critical arrhythmia may be sent over the cellular network. In one embodiment, the signals sent over the cellular network may be sent redundantly. The monitoring device 103 may include a processor adapted to detect critical arrythmias patterns in the data, which need to be transmitted.

In one possible embodiment, when a critical arrhythmia is identified or detected, the monitoring device 103 may transmit a low data size data string containing the critical arrhythmia ECG data. The critical arrhythmia ECG data may be sent in one or more than one format. The recipient of the critical arrhythmia ECG data may compare and combine multiple ECG data packages to assemble one lossless ECG data strip from the multiple transmitted messages. The possible formats for transmission may include:
a. Main cellular 4G data format and transmission protocol;
b. Main cellular 5G data format and transmission protocol;
c. Utilization of open WIFI connections, such as, but not limited to, a nationwide cable or Internet provider;
d. Main cellular 4G data format and transmission protocol on a medical use channel;
e. Main cellular 5G data format and transmission protocol on a medical use channel; and
f. Preconnected, preauthorized, or open connections including, by way of example, Bluetooth, ZIGBEE, NF, or other mesh networks of home automation hub connections such as cellular phones, cellular hotspots provided by personal devices, power meters, Google Home devices, Apple devices, Alexa devices, automobiles, aircrafts, or the like.

The selective send protocol may be particularly beneficial in embodiments in which the human body detunes the antenna 104 of the heart monitoring device 103 or otherwise reduces transmission efficiencies in ways that may not be overcome due to SAR limitations. The monitoring device 103 may include a processor adapted to execute an algorithm, which may detect anomalies, such as, but not limited to, atrial fibrillation, in the data. In such an embodiment, only a limited portion of the data identified as containing an anomaly may be selected for transmission. Sending only limited data, which may be critical arrhythmias, uses less battery power because only a subset of ECG data, rather than all of the ECG data, is transmitted by the heart monitoring device 103. In such an embodiment, portions of data surrounding the anomaly may also be transmitted. In such an embodiment, the cellular enabled chest worn heart monitor 100 may selectively transmit all or only a subset of data to a monitoring center for further analysis.

In another possible embodiment, a selective send for SAR or pacemaker protocol may be utilized. Such an embodiment may operate similarly to the selective send embodiment and have additional capabilities. In a selective send for SAR or pacemaker, the heart monitoring device 103 may detect the electrode 101 impedance to determine whether or not the heart monitor 100 is worn by the patient. The heart monitor may transmit full packages only when it is not worn by the patient. When the heart monitor 100 is worn by the patient, only short data messages with critical arrhythmias may be transmitted. In such an embodiment, a reflective power measurement may be taken at the antenna to optimize the efficiency and SAR characteristics of the monitor 100. This optimization may be performed by peak detection and onboard S11 and S21 measurements and comparisons.

In one embodiment, data may be transmitted over SMS data channels.

Another protocol may always send the same data while varying transmission or reception power based upon whether or not the heart monitor 100 is worn by a patient.

In embodiments in which data transmission is related to whether or not the monitoring device 103 is worn by the patient, the monitoring device 103 may notify the patient when there is data to be transmitted and instruct the patient to remove the monitoring device 103 from the patient's body and place the monitoring device 103 a safe distance away from the patient prior to transmitting data. The monitoring device 103 may automatically begin transmission when it detects the conditions are safe to do so or the patient may provide an input to the monitoring device 103 signaling that conditions are safe for transmission. Upon completion of transmission, the monitoring device 103 may notify the patient that transmission is complete. Upon receipt of this notification, the patient may secure the monitoring device 103 to the patient's body.

Additional strategies may be implemented by the monitoring device 103 to reduce power consumption, including, but not limited to removing any ECG waveform detection algorithm, or a portion of any ECG waveform detection algorithm, from the monitoring device 103. In such an embodiment, the monitoring device 103 may record the ECG signal and transmit all ECG signals to a monitoring center. The ECG signals may be provided to proprietary algorithms and analyzed at the monitoring center rather than by the monitoring device 103 to conserve power usage of the monitoring device 103.

In one embodiment, power usage by the monitoring device 103 may be reduced by utilizing one or more data compression algorithms to increase the efficiency of data transmission.

In one embodiment, one or more data signals may be transmitted by the monitoring device 103 in low fidelity, which may be accomplished, by way of example, and not as a limitation, by utilizing a high loss compression algorithm. The data may be initially reviewed by the monitoring center with one or more messages sent back to the monitoring device 103 to request higher fidelity data if higher fidelity data is desirable.

In one embodiment, ECG data may be sent by the monitoring device 103 in near real time or in various size packets throughout the monitoring procedure.

In one embodiment, which may be used in combination with the aforementioned transmission protocols. SAR may be limited by using a ground plane on the heart monitoring device 103 or elsewhere in the housing, which is in electrical connection with the antenna 104. The ground plane may also be in electrical communication with one or more electrodes 101.

In one embodiment, the antenna 104 may be placed inside a reusable material, which, by way of example and not as a limitation, may be silicone or the like. The reusable material may be adapted to secure or adhere to the patient's body. Such an embodiment may allow the antenna 104 to be farther from the patient's body.

The antenna 104 may be placed on the top or front of the monitoring device 103 to create maximum distance from the body. In such an embodiment, the antenna 104 could be incorporated into the housing of the heart monitoring device 103 or embedded in a silicone or like material.

The housing, antenna 104, or the location at which the antenna is secured to the monitoring device 103 may be shielded to increase protection from SAR.

In one embodiment, the antenna 104 may be integrated into the housing of the monitoring device 103. The monitoring device 103 housing may include a retractable section, which may be affixed to at least a portion of the antenna 104. The retractable section may be extended away from the monitoring device 103 housing from a retracted position to an extended position to effectively increase the size of the antenna 104 as compared to when the retractable section is in a retracted position.

In still another embodiment, which, again, may be used in combination with other embodiments contained herein, a docking station 108 may be utilized as a mobile port or dock. The docking station 108 may be adapted to receive the monitoring device 103 and charge a battery carried by the monitoring device 103. The docking station 108 may have the capability of sending on a different network than the heart monitoring device 103 or the docking station 108 may have more reliable network coverage than the heart monitoring device 103. Such an embodiment may require the docking station 108 to have cellular capabilities that duplicate or enhance those of the heart monitoring device 103. Such an embodiment could be used to send full data packages when the heart monitoring device 103 comes within a distance of the docking station 108, which allows a Bluetooth connection to be established. The monitoring device 103 may be capable of detecting when it is carried by the docking station 108 and only enable transmission in such circumstances. The docking station 108 may include an antenna, battery, or other power source. In one embodiment, the docking station 108 may include a cellular transmission system. In such an embodiment, the monitoring device 103 may provide data to the docking station, which may then transmit the data on a cellular network. Such an embodiment could be used in combination with the selective send protocol to allow critical arrhythmias to be transmitted as identified or detected by the monitoring device 103 while full data packages may only be sent by the docking station 108. Such an embodiment would reduce power and SAR impacts of the heart monitoring device 103. The docking station 108 may use Bluetooth, WIFI, NF, Zigbee, or other mesh communication to receive data from the heart monitoring device 103. WIFI or cellular communication protocols could then be utilized by the docking station 108 to transmit the data to a base station or monitoring center.

In one embodiment, a reusable and rechargeable battery may be used by the monitoring device 103. Such a battery may be easily swapped out for a fresh battery when power is depleted. The battery may be replaced with or without removing the monitoring device 103 from the patient's body. In embodiments in which the monitoring device 103 must be removed to change the battery, the battery swap process may be completed quickly and easily by the patient with minimal interruption to the monitoring procedure.

In one embodiment, multiple batteries may be utilized by the monitoring device 103 to support the power requirements of the cellular enabled chest worn heart monitor 100 device.

In one embodiment, a patient may be provided with a plurality of monitors 100, each incorporating a monitoring device 103. Each of the monitors 100 may be easily removable and capable of magnetically charging while another of the plurality of monitors 100 is worn by the patient. Both monitors 100 and corresponding monitoring devices 103 may be triaged to the same procedure and data continuity may be managed by the monitoring center using software that supports the monitoring devices 103.

The heart monitor 100 may optionally have encryption wrapper and VPN capability to encrypt data sent from the heart monitor 100 to the cloud during data transmission.

The heart monitor 100 may include a GPS chipset and be configured to send a current GPS location. Software or firmware of the heart monitor 100 may be updated Over the Air (OTA).

The heart monitor 100 may include a voice recognition module, which may provide voice capabilities, including, but not limited to, voice over LTE (VoLTE). In one embodiment, a patient may be able to speak to the heart monitor 100 about symptoms in addition to or in lieu of pressing a symptom button. In embodiments in which VoLTE offers speech recognition, the heart monitor 100 may receive simple commands from the patient, including, but not limited to, receiving a patient query. In one embodiment, the patient may audibly query the heart monitor 100 for the status of the battery life and may receive an audible response from the heart monitor 100 providing the current battery level, projected time left until battery depletion, or the like.

The heart monitor 100 may be paired with one or more external devices to facilitate sending physiological data to a clinical monitoring center. Some of the illustrative aspects of the present invention may be advantageous in solving the problems herein described and other problems not discussed which are discoverable by a skilled artisan.

While the above description contains much specificity, these should not be construed as limitations on the scope of any embodiment, but as exemplifications of the presented embodiments thereof. Many other ramifications and variations are possible within the teachings of the various embodiments. While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best or only mode contemplated for carrying out this invention, but that the invention will include all embodiments falling within the description of the invention. Also, in the drawings and the description, there have been disclosed exemplary embodiments of the invention and, although specific terms may have been employed, they are unless otherwise stated used in a generic and descriptive sense only and not for purposes of limitation, the scope of the invention therefore not being so limited. Moreover, the use of the terms first, second, etc. do not denote any order or importance, but rather the terms first, second, etc. are used to distinguish one element from another. Furthermore, the use of the terms a, an, etc. do not denote a limitation of quantity, but rather denote the presence of at least one of the referenced item.

What is claimed is:

1. A system for physiological signal monitoring comprising:
a housing;
a physiological signal sensor carried by the housing;
a microphone carried by the housing; and
a symptom button carried by the housing; and
wherein the microphone is operable to capture an audio recording upon activation of the symptom button and a physiological signal captured by the physiological signal sensor during the activation of the symptom button is associated with the audio recording.
